(19) United States
(12) Patent Application Publication
(10) Pub. No.: US 2023/0363772 A1
(43) Pub. Date: Nov. 16, 2023

(54) SYSTEM AND METHOD FOR LOCKING AN INTRAMEDULLARY NAIL IN A BONE

(71) Applicant: OFEK ESHKOLOT RESEARCH AND DEVELOPMENT LTD., Karmiel (IL)

(72) Inventors: Orit BRAUN BENYAMIN, Yuvalim (IL); Idan ILLOUZ, Naharia (IL); Ravid MOR-YOSSEF, Ramat-Yishai (IL)

(21) Appl. No.: 18/030,063

(22) PCT Filed: Oct. 7, 2021
(86) PCT No.: PCT/IL2021/051205

§ 371 Date: Apr. 3, 2023

Related U.S. Application Data
(60) Provisional application No. 63/089,031, filed on Oct. 8, 2020.

(51) Int. Cl. A61B 17/17

(52) U.S. Cl. CPC A61B 17/1725; A61B 17/1703; A61B 17/7241

(57) ABSTRACT

The present subject matter provides a system for inserting a locking screw into a nail hole of an intramedullary nail inserted in a medullary canal of a bone, the system comprising: a trocar having a tube like structure that is open at both sides; a drill centralizer having an elongated shape and comprising a sharp centralizer tip configured to puncture the bone; a sleeve having a tube-like structure that is open at both sides; a guide having a tube-like structure that is open at both sides; and a drill bit of a drilling device, comprising a drill bit tip. A method for inserting a locking screw into a nail hole of an intramedullary nail inserted in a medullary canal of a bone, using the system, as well as various embodiments of the system and method, are disclosed as well

### CROSS-REFERENCE TO RELATED APPLICATION

This application claims priority to U.S. Provisional Pat. Application No. 63/089,031, filed Oct. 08, 2021, the entire contents of which is incorporated herein by reference in its entirety.

### FIELD

The present subject matter relates to intramedullary nailing of a bone. More particularly, the present subject matter relates to inserting locking screws into holes of an intramedullary nail during intramedullary nailing of a bone.

### BACKGROUND

Intramedullary nailing is a process of fixing a broken bone having an intramedullary canal. During intramedullary nailing, it is a common practice to initially insert into the medullary canal, from a proximal end of the medullary canal, a ball-ended guide wire, and then pass the ball-ended guide wire down the canal toward a distal fracture fragment of the broken bone. Then, the inner diameter of the medullary canal is gradually increased by passing in succession a series of flexible intramedullary reamers having increasing diameters over the guide wire. The guide wire is configured not only to guide the passage of the flexible intramedullary reamers through the intramedullary canal, but also to allow withdrawal of a tip of a reamer in the unlikely case that the reamer breaks during reaming. Once reaming is completed, the ball-ended guide wire is usually replaced with a smooth-tipped guide wire, and then a hollow intramedullary nail is inserted over the smooth-tipped guide wire. The intramedullary nail has at least one proximal hole and at least one distal hole. These holes are configured to receive locking screws in order to fix the intramedullary nail in place, and to provide secure attachment between the intramedullary nail and the most proximal and distal fracture fragments of the broken bone. Then, once successful positioning of the intramedullary nail is achieved, the smooth-tipped guide wire is withdrawn, and proximal and distal locking screws are inserted into the proximal and distal holes of the intramedullary nail through drills that are made in the bone tissue.

A common challenge in the process of intramedullary nailing is the positioning of the exact location of the holes of the intramedullary nail that is inserted in the medullary canal. This is necessary in order to drill the bone tissue over the exact location of the holes of the intramedullary nail, in order to allow successful insertion of the locking screws into the holes of the intramedullary nail. Positioning of the at least one proximal hole of the intramedullary nail is relatively straightforward, because during operation there is a possibility to design fixtures that can be used as drill guides for positioning of the at least one proximal hole of the intramedullary nail.

However, positioning of the at least one distal hole of the intramedullary nail is more challenging, because during insertion of the intramedullary nail into the medullary canal, some twisting and bending of the intramedullary nail can occur, in addition to the natural curvature of the medullary canal that cannot be predicted. As a result, it is impossible to position with certainty the location of the at least one distal hole of the medullary nail only by relying on geometric relationship between the proximal and distal ends of the intramedullary nail. As a result, there is a necessity to perform X-ray imaging, usually many times, in order to exactly position the at least one distal hole of the intramedullary nail before drilling the drills for the locking screws. This practice exposes a patient that undergoes intramedullary nailing, and a medical staff that performs the intramedullary nailing, to excessive ionizing irradiation, particularly X-ray irradiation. This ionizing irradiation should be reduced to minimal, or preferably completely avoided, during intramedullary nailing.

### SUMMARY

Unless otherwise defined, all technical and scientific terms used herein have the same meaning as commonly understood by one of ordinary skill in the art to which this subject matter belongs. Although methods and materials similar or equivalent to those described herein can be used in the practice or testing of the present subject matter, suitable methods and materials are described below. In case of conflict, the patent specification, including definitions, will control. In addition, the materials, methods, and examples are illustrative only and not intended to be limiting.

According to one aspect of the present subject matter, there is provided a system for inserting a locking screw into a nail hole of an intramedullary nail inserted in a medullary canal of a bone, the system comprising:
a trocar having a tube like structure that is open at both sides, configured to attach to a bone;
a drill centralizer having an elongated shape and comprising a sharp centralizer tip configured to puncture the bone, wherein the drill centralizer is configured to be inserted into the trocar and puncture the bone and create a starter hole in the bone;
a sleeve having a tube-like structure that is open at both sides, configured to be inserted into the trocar, and guide a camera to a center of a drill drilled in the bone;
a guide having a tube-like structure that is open at both sides, configured to be inserted into the trocar, accommodate a drill bit and guide the drill bit to the starter hole punctured on the surface of the bone, and accommodate the drill bit during the drilling in the bone; and
a drill bit of a drilling device, wherein the drill bit comprising a drill bit tip configured to be in contact with the bone surface at an early stage of drilling, to be accommodated in the start hole punctured in the bone at the beginning of the drilling, wherein the drill bit is configured to drill a hole in the bone when the drill bit tip points toward the bone.

According to one embodiment, at least an inner space of the trocar is sterile.

According to another embodiment, wherein the drill centralizer is sterile.

According to yet another embodiment, at least an inner space of the guide is sterile.

According to still another embodiment, the drill bit is sterile.

According to a further embodiment, the sleeve is not sterile.

According to yet a further embodiment, the sleeve is sterile.

According to still a further embodiment, an inner diameter of the sleeve is fit to a diameter of the camera.

According to an additional embodiment, the sleeve further comprises at least one notch along an inner part of the sleeve, wherein the at last one notch is configured to fit to an at least one protrusion of the camera, and wherein the at least one notch is configured to circularly fix the camera and prevent rotation of the camera around a longitudinal axis of the camera.

According to yet an additional embodiment, the at least one notch is configured to prevent rotation of the camera around a longitudinal axis of a rod or wire to which the camera is attached.

According to still an additional embodiment, the at least one notch is configured to allow sliding of the protrusion of the camera along the notch.

According to an additional embodiment, the at least one notch runs from an external edge of the sleeve, and ends at a distance from an internal edge of the sleeve.

According to yet another embodiment, the distance from an internal edge of the sleeve is configured to prevent contact of the camera with the bone and tissues in the vicinity of the bone.

According to another embodiment, the guide is further configured to accommodate a locking screw and guide the locking screw to the drill made in the bone and further into the nail hole that is aligned with the drill made in the bone.

According to another aspect of the present subject matter, there is provided a method for inserting a locking screw into a nail hole of an intramedullary nail inserted in a medullary canal of a bone, the method comprising:
providing a bone with a guide wire inserted into a medullary canal of the bone, after the medullary canal was expanded;
providing a system as described herein;
substantially vertically attaching the trocar to the bone in a location where it is desired to insert a locking screw into a nail hole of an intramedullary nail that is inserted into the intramedullary canal of the bone;
inserting the drill centralizer into the trocar;
puncturing a starter hole in the bone with the centralizer tip of the drill centralizer;
removing the drill centralizer from the trocar;
inserting the guide into the trocar;
inserting the drill bit of the drilling device into the guide;
drilling a drill in the bone until the medullary canal of the bone;
removing the drill bit from the guide;
removing the guide from the trocar;
inserting the sleeve into the trocar;
inserting the camera into the sleeve;
acquiring visual light images of the medullary canal through the drill in the bone with the camera;
inserting the intramedullary nail into the medullary canal of the bone, through the guide wire that is inserted in the medullary canal, until a nail hole of the intramedullary nail is detected by the camera;
aligning the nail hole of the intramedullary nail with the drill made in the bone;
removing the camera from the sleeve;
removing the sleeve from the trocar;
inserting the guide into the trocar;
inserting the locking screw into the screw guide;
inserting the locking screw into the nail hole of the intramedullary nail that is aligned with the drill in the bone; and
removing the trocar, and the screw guide from the bone.

According to still another aspect of the present subject matter, there is provided a system for use in inserting a locking screw into a nail hole of an intramedullary nail inserted in a medullary canal of a bone, the inserting of the locking screw into the nail hole of the intramedullary nail inserted in the medullary canal of the bone, comprising:
providing a bone with a guide wire inserted into a medullary canal of the bone, after the medullary canal was expanded;
providing a system as described herein;
substantially vertically attaching the trocar to the bone in a location where it is desired to insert a locking screw into a nail hole of an intramedullary nail that is inserted into the intramedullary canal of the bone;
inserting the drill centralizer into the trocar;
puncturing a starter hole in the bone with the centralizer tip of the drill centralizer;
removing the drill centralizer from the trocar;
inserting the guide into the trocar;
inserting the drill bit of the drilling device into the guide;
drilling a drill in the bone until the medullary canal of the bone;
removing the drill bit from the guide;
removing the guide from the trocar;
inserting the sleeve into the trocar;
inserting the camera into the sleeve;
acquiring visual light images of the medullary canal through the drill in the bone with the camera;
inserting the intramedullary nail into the medullary canal of the bone, through the guide wire that is inserted in the medullary canal, until a nail hole of the intramedullary nail is detected by the camera;
aligning the nail hole of the intramedullary nail with the drill made in the bone;
removing the camera from the sleeve;
removing the sleeve from the trocar;
inserting the guide into the trocar 12;
inserting the locking screw into the screw guide;
inserting the locking screw into the nail hole of the intramedullary nail that is aligned with the drill in the bone; and
removing the trocar, and the screw guide from the bone.

According to one embodiment, the aligning the nail hole of the intramedullary nail with the drill made in the bone is achieved by rotating the intramedullary nail around a longitudinal axis of the intramedullary nail.

According to another embodiment, the nail hole of the intramedullary nail is a distal nail hole, wherein the locking screw is firstly inserted into the distal nail hole of the intramedullary nail, and then, a locking screw is inserted into a proximal nail hole of the intramedullary nail.

### BRIEF DESCRIPTION OF THE DRAWINGS

Embodiments are herein described, by way of example only, with reference to the accompanying drawings. With specific reference now to the drawings in detail, it is stressed that the particulars shown are by way of example and for purposes of illustrative discussion of the preferred embodiments, and are presented in the cause of providing what is believed to be the most useful and readily understood description of the principles and conceptual aspects of the embodiments. In this regard, no attempt is made to show structural details in more detail than is necessary for a fundamental understanding, the description taken with the drawings making apparent to those skilled in the art how several forms may be embodied in practice.

In the drawings:

FIG. 1 schematically illustrates, according to an exemplary embodiment, a lateral view of a bone, a trocar attached to the bone, and a drill centralizer aside the trocar.

FIG. 2 schematically illustrates, according to an exemplary embodiment, a lateral view of a bone, a trocar attached to the bone, and a drill centralizer inserted in the trocar.

FIG. 3, schematically illustrates, according to an exemplary embodiment, a bone, a trocar attached to the bone, and a guide aside the trocar.

FIG. 4, schematically illustrates, according to an exemplary embodiment, a lateral view of a bone, a trocar attached to the bone, and a guide inserted in the trocar.

FIG. 5, schematically illustrates, according to an exemplary embodiment, a bone, a trocar attached to the bone, a guide inserted in the trocar, and a drill bit aside the trocar.

FIG. 6, schematically illustrates, according to an exemplary embodiment, a lateral view of a bone, a trocar attached to the bone, a guide inserted in the trocar, and a drill bit inserted in the trocar and drilling a hole in the bone.

FIG. 7 schematically illustrates, according to an exemplary embodiment, a lateral view of a bone, a trocar attached to the bone, and a sleeve aside the trocar.

FIG. 8 schematically illustrates, according to an exemplary embodiment, a lateral view of a bone, a trocar attached to the bone, and a sleeve inserted in the trocar.

FIG. 9, schematically illustrates, according to an exemplary embodiment, a frontal view of a bone, a trocar attached to the bone, and a sleeve inserted in the trocar 12.

FIG. 10, schematically illustrates, according to an exemplary embodiment, a detailed frontal view of a sleeve inserted in a trocar that is attached to a bone.

FIG. 11 schematically illustrates, according to an exemplary embodiment, a perspective see-through view of a bone, an intramedullary nail inserted into a medullary canal of the bone, a trocar attached to the bone, and a guide inserted in the trocar.

FIG. 12, schematically illustrates, according to an exemplary embodiment, a lateral see-through view of a bone, an intramedullary nail inserted into a medullary canal of the bone, a trocar attached to the bone, a guide inserted in the trocar, and a locking screw screwed into a nail hole of the intramedullary nail through the guide.

### DESCRIPTION OF THE PREFERRED EMBODIMENTS

Before explaining at least one embodiment in detail, it is to be understood that the subject matter is not limited in its application to the details of construction and the arrangement of the components set forth in the following description or illustrated in the drawings. The subject matter is capable of other embodiments or of being practiced or carried out in various ways. Also, it is to be understood that the phraseology and terminology employed herein is for the purpose of description and should not be regarded as limiting. In discussion of the various figures described herein below, like numbers refer to like parts. The drawings are generally not to scale.

For clarity, non-essential elements were omitted from some of the drawings.

In one aspect, the present subject matter provides a system for inserting a locking screw into a hole of an intramedullary nail inserted in a medullary canal of a bone. In another aspect, the present subject matter provides a method for inserting a locking screw into a hole of an intramedullary nail inserted in a medullary canal of a bone.

The system and method for inserting a locking screw into a hole of an intramedullary nail inserted in a medullary canal of a bone of the present subject allow insertion of the locking screw into the hole of the intramedullary nail inserted in the medullary canal of the bone precisely and without exposing the patient undergoing the intramedullary nailing, and the medical staff that performs the intramedullary nailing, to ionizing irradiation, particularly X-ray irradiation. Prior art procedures include firstly inserting the intramedullary nail in the medullary canal, and then attempting to position the at least one distal hole of the intramedullary nail, either by assumption or by X-ray imaging, and only then drilling in the bone a pass to an at least one locking screw in the location that was determined previously in order to allow insertion of the at least one locking screw into its corresponding at least one hole in the intramedullary nail.

On the other hand, the system and method of the present subject matter provide a different strategy for inserting a locking screw into a hole of an intramedullary nail inserted in a medullary canal of a bone. At first, after an intramedullary nail is inserted into the medullary canal of the bone, a drill is made in the bone in an estimated location of the hole of the intramedullary nail. The estimation of the location of the hole of the intramedullary nail can be performed for example by X-ray imaging, or by estimation according to the geometry of the bone and the intramedullary nail, and the like. Then, the orientation and location of the intramedullary nail in the medullary canal are changed in a manner that brings the hole of the intramedullary nail to the exact position of the drill, in order to allow precise insertion of the locking screw into the hole through the drill. Thus, the present subject matter entirely changes the concept of intramedullary nailing.

The present subject matter provides a system 1 for inserting a locking screw into a nail hole of an intramedullary nail inserted in a medullary canal of a bone 500, the system 1 comprises:
a trocar 12 having a tube like structure that is open at both sides, configured to attach to a bone 500;
a drill centralizer 14 having an elongated shape and comprising a sharp centralizer tip 142 configured to puncture the bone 500, wherein the drill centralizer 14 is configured to be inserted into the trocar 12 and puncture the bone 500 and create a starter hole in the bone 500;
a sleeve 16 having a tube-like structure that is open at both sides, configured to be inserted into the trocar 12, and guide a camera to a center a drill drilled in the bone 500;
a guide 18 having a tube-like structure that is open at both sides, configured to be inserted into the trocar 12, accommodate a drill bit 19 and guide the drill bit 19 to the starter hole punctured on the surface of the bone 500, and accommodate the drill bit 19 during the drilling in the bone 500; and
a drill bit 19 of a drilling device, wherein the drill bit 19 comprising a drill bit tip 192 configured to be in contact with the bone 500 surface at an early stage of drilling, to be accommodated in the start hole punctured in the bone 500 at the beginning of the drilling, wherein the drill bit 19 is configured to drill a hole in the bone 500 when the drill bit tip 192 points toward the bone 500.

For the sake of simplicity, the term “system 1” relates to the system 1 for inserting a locking screw into a hole of an intramedullary nail inserted in a medullary canal of a bone. The components of the system 1 and their functions are described in detail hereinafter.

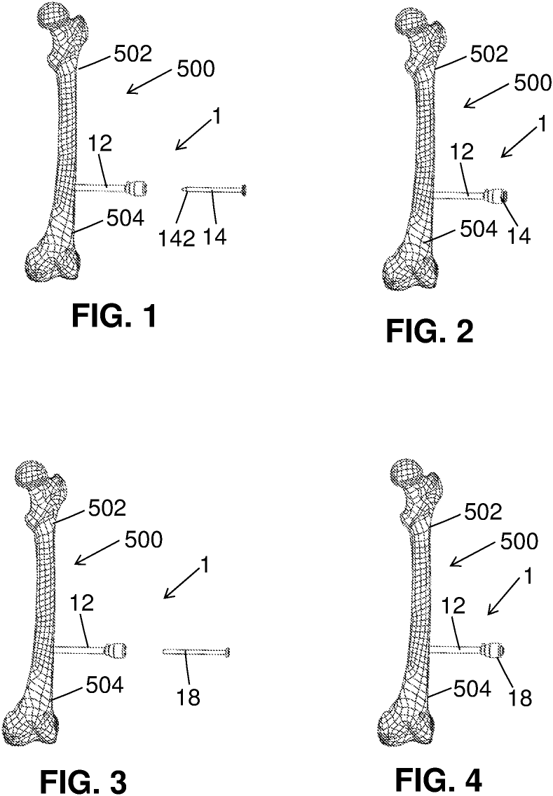

Referring now to FIG. 1, schematically illustrating, according to an exemplary embodiment, a lateral view of a bone 500, a trocar 12 attached to the bone 500, and a drill centralizer 14 aside the trocar 12. The bone 500 is, for example, a long bone 500, which has a proximal end 502 and a distal end 504. An intramedullary nail has to be inserted into the medullary canal of the bone 500 in order to fix the bone 500, for example when the bone 500 is broken. As mentioned above, the intramedullary nail has to be fixed by inserting locking nails into holes of the intramedullary nail. This is done by drilling the bone 500 precisely at the locations of the holes, in order to allow precise insertion of the locking nails into the holes of the intramedullary nail. However, since the intramedullary nail is inserted in the medullary canal of the bone 500, positioning of the holes of the intramedullary nail is challenging, when there is a desire to eliminate exposure of the patient and the medical staff to ionizing irradiation, particularly X-ray irradiation, because the holes are not seen from outside the bone 500, and because assumption of the position of the holes according to geometric relations between the holes and the structure of the intramedullary nail is not precise because of reasons mentioned above. The present subject matter provides a solution to this challenge.

FIG. 1 further illustrates a trocar 12 substantially attached to the bone 500. The trocar 12 can be held in place, while attached to the bone 500, for example by manually fixing the trocar 12 in place, or preferably by using a fixation tool. The trocar 12 is attached to the bone 500 in a place where there is a desire to drill a drill for inserting a locking screw into a hole of the intramedullary nail that is inserted in the medullary canal of the bone 500. The trocar 12 illustrated in FIG. 1 is attached substantially at the distal end 504 of the bone 500. However, this location of the attachment of the trocar 12 to the bone 500 is only exemplary. The trocar 12 can be attached at any location on the bone 500, where there is a desire to drill a drill for a locking screw, for example substantially at the proximal end 502 of the bone 500 or at any other location along the bone 500. Nevertheless, the trocar 12 is preferably attached substantially at the distal end 504 of the bone 500, because positioning of the at least one distal hole of the intramedullary nail is most challenging. According to one embodiment, the trocar has a tube-like structure that is open at both sides.

Also illustrated in FIG. 1 is a drill centralizer 14 aside the trocar 12. The drill centralizer 14 is configured to be inserted into the trocar 12, and puncture the bone 500 at a location where there is a desire to drill a hole in the bone. According to one embodiment, drill centralizer 14 has an elongated shape and a sharp centralizer tip 142 configured to puncture the bone 500 and create a starter hole in the bone 500. Thus, when the drill centralizer 14 is inserted into the trocar 12, the orientation of the drill centralizer 14 is such that the centralizer tip 142 points toward the bone 500, as illustrated in FIG. 1. Since the drill centralizer 14 is configured to be in contact with a tissue, i.e. the bone 500 tissue, then according to one embodiment, at least an inner space of the trocar 12 is sterile, and according to another embodiment, the drill centralizer 14 is sterile.

Referring now to FIG. 2, schematically illustrating a lateral view of a bone 500, a trocar 12 attached to the bone 500, and a drill centralizer 14 inserted in the trocar 12. At this stage, the centralizer tip 142 is in contact with a surface of the bone 500 and punctures the starter hole in the bone 500. The starter hole that is punctured by the centralizer tip 142 is configured to receive a tip of the drill bit when the bone 500 is drilled and hold the tip of the drill bit at the beginning of the drilling. After the starter hole is punctured, the drill centralizer 14 removed from the trocar 12.

Referring now to FIG. 3, schematically illustrating, according to an exemplary embodiment, a bone 500, a trocar 12 attached to the bone 500, and a guide 18 aside the trocar 12. According to one embodiment, the guide 18 has a tube-like structure that is open at both sides. According to another embodiment, the guide 18 is configured to be inserted into the trocar 12. According to yet another embodiment, the guide 18 is configured to accommodate a drill bit 19 and guide the drill bit 19 to the starter hole punctured on the surface of the bone 500. The guide 18 is also configured to accommodate the drill bit 19 during the drilling in the bone 500.

Referring now to FIG. 4, schematically illustrating, according to an exemplary embodiment, a lateral view of a bone 500, a trocar 12 attached to the bone 500, and a guide 18 inserted in the trocar 12. At this stage, the guide 18 is inserted in the trocar 12.

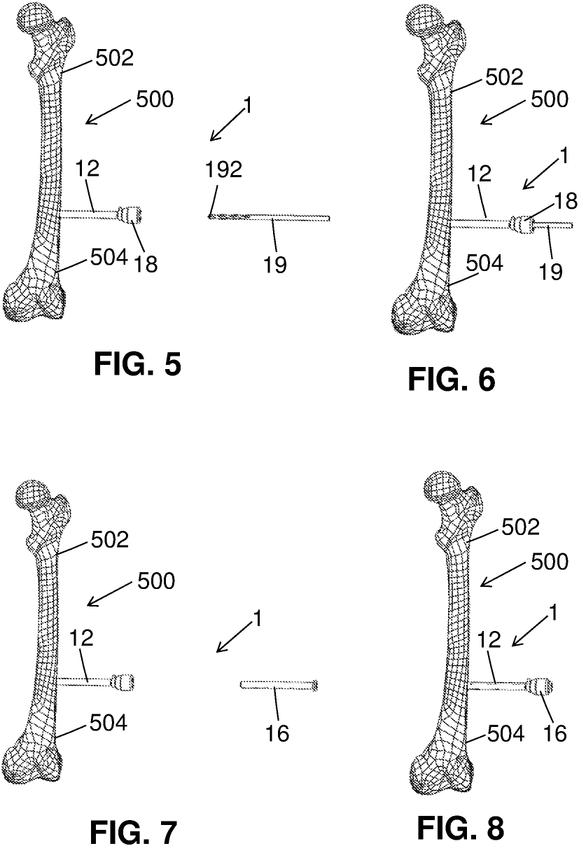

Referring now to FIG. 5, schematically illustrating, according to an exemplary embodiment, a bone 500, a trocar 12 attached to the bone 500, a guide 18 inserted in the trocar 12, and a drill bit 19 aside the trocar 12. According to one embodiment, the drill bit 19 comprises a drill bit tip 192 configured to be in contact with the bone 500 surface at an early stage of drilling. According to another embodiment, the drill bit tip 192 is configured to be accommodated in the start hole punctured in the bone 500 at the beginning of the drilling. According to yet another embodiment, the drill bit 19 is configured to drill a hole in the bone 500. Thus, when the drill bit 19 is inserted into the guide 18, the orientation of the drill bit 19 is such that the drill bit tip 192 points toward the bone 500, as illustrated in FIG. 5. Since during drilling there is contact with the bone 500 tissue, then according to one embodiment, at least an inner space of the guide 18 is sterile, and according to another embodiment, the drill bit 19 is sterile.

Referring now to FIG. 6, schematically illustrating, according to an exemplary embodiment, a lateral view of a bone 500, a trocar 12 attached to the bone 500, a guide 18 inserted in the trocar 12, and a drill bit 19 inserted in the trocar 12 and drilling a hole in the bone 500. At this stage the drill bit 19 drills a hole in the bone 500. FIG. 6 also illustrates a see-through image of the bone 500 showing the drill bit tip 192 drilling into the bone 500 tissue.

Referring now to FIG. 7, schematically illustrating, according to an exemplary embodiment, a bone 500, a trocar 12 attached to the bone 500, and a sleeve 16 aside the trocar 12. After a drill is made in the bone 500, the drill bit 19 is removed from the guide 18, and the guide 18 is removed from the trocar 12. Then, the sleeve 16 is to be inserted into the trocar 12. The sleeve 16 has a tube-like structure that is open at both sides, and is configured to be inserted into the trocar 12.

Referring now to FIG. 8, schematically illustrating, according to an exemplary embodiment, a lateral view of a bone 500, a trocar 12 attached to the bone 500, and a sleeve 16 inserted in the trocar 12. At this stage the sleeve 16 is inserted in the trocar 12. According to one embodiment, the sleeve 16 is configured to guide a camera to a center of the drill in the bone 500. The sleeve 16 is configured to guide a camera that is attached to a guiding rod or a guiding wire, for example a camera similar to a laparoscopic camera. In a preferred embodiment, the camera is a visual light camera. Therefore, the camera can further comprise a visual light source. In some embodiments, when the camera is not supposed to touch a tissue, for example the bone 500 tissue, the camera is not sterile. Therefore, according to these embodiments, the sleeve 16 is not sterile as well. However, in some other embodiments, the sleeve 16 is sterile, for example to avoid contamination of the bone 500 tissue.

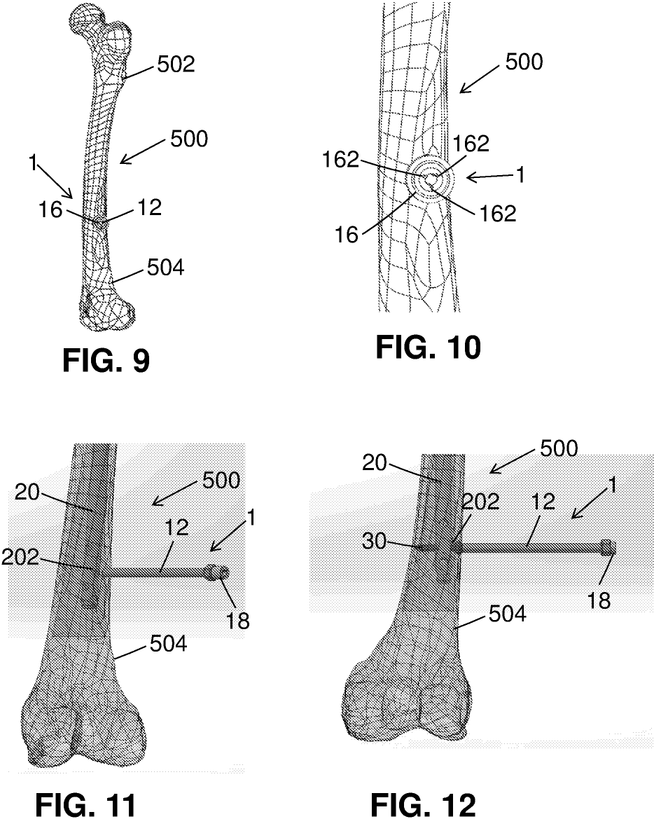

Referring now to FIG. 9, schematically illustrating, according to an exemplary embodiment, a frontal view of a bone 500, a trocar 12 attached to the bone 500, and a sleeve 16 inserted in the trocar 12; and to FIG. 10, schematically illustrating, according to an exemplary embodiment, a detailed frontal view of a sleeve 16 inserted in a trocar 12 that is attached to a bone 500. As mentioned above, the sleeve 16 is configured to guide a camera to a center of the drill in the bone 500. This embodiment can be achieved for example by fitting an inner diameter of the sleeve 16 to a diameter of the camera, in order to fix the camera in place. In other words, according to one embodiment, an inner diameter of the sleeve 16 is fit to a diameter of the camera. However, this embodiment does not prevent rotation of the camera around a longitudinal axis of the rod or wire to which the camera is attached. Therefore, according to another embodiment, the sleeve 16 further comprises at least one notch 162 along an inner part of the sleeve 16, wherein the at last one notch 162 is configured to circularly fit to an at least one protrusion of the camera. The at least one notch 162 is configured to circularly fix the camera and prevent rotation of the camera around a longitudinal axis of the camera. In other embodiments, the at least one notch 162 is configured to prevent rotation of the camera around a longitudinal axis of a rod or wire to which the camera is attached. Furthermore, the notch 162 is also configured to allow sliding of the protrusion of the camera along the notch 162.

According to one embodiment, the at least one notch 162 runs from an external edge of the sleeve 16, and ends at a distance from an internal edge of the sleeve 16. An exemplary aim of this embodiment, is to prevent pushing of the camera further in the sleeve 16, and as a result, prevent contact of the camera with the bone and tissue in the vicinity of the bone, for example in order to prevent damage or contamination of the bone by the camera. Thus, according to another embodiment, the at least one notch 162 ends at a distance from the internal edge of the sleeve 16 that is configured to prevent contact of the camera with the bone and tissues in the vicinity of the bone.

The sleeve 16, shown in FIG. 10, comprises an exemplary number of three notches 162 in a certain configuration. Therefore, the camera can comprise three protrusions in a similar configuration that fit the three notches 162. It should be noted that the sleeve 16 comprising the three notches 162, which is illustrated in FIG. 10, is only exemplary, and should not be considered as limiting the scope of the present subject matter.

When the camera is inserted in the sleeve 16, visual light images of the medullary canal of the bone 500 are acquired while an intramedullary nail is inserted into the medullary canal, until a nail hole of the intramedullary nail is detected by the camera. At this stage, the nail hole is aligned with the drill made in the bone 500. This can be achieved, for example, by rotating the intramedullary nail around a longitudinal axis of the intramedullary nail, while observing the visual light images that are acquired by the camera that is inserted in the sleeve 16. After the nail hole is aligned with the drill, the camera is removed from the sleeve 16, and the sleeve 16 is removed from the trocar 12.

Referring now to FIG. 11 schematically illustrating, according to an exemplary embodiment, a perspective see-through view of a bone 500, an intramedullary nail 20 inserted into a medullary canal of the bone 500, a trocar 12 attached to the bone 500, and a guide 18 inserted in the trocar 12. At this stage a nail hole 202 of the intramedullary nail 20 is aligned with the drill made in the bone 500. After the camera and the sleeve 16 were removed from the trocar 12, the guide 18 is inserted again into the trocar 12. It was mentioned above that the guide 18 is configured to accommodate the drill bit 19 and guide the drill bit 19 to the starter hole punctured on the surface of the bone 500. According to another embodiment, the guide 18 is further configured to accommodate a locking screw and guide the locking screw to the drill made in the bone 500 and further into the nail hole 202 that is aligned with the drill made in the bone 500. Since the locking screw has to be sterile, because it is to be in contact with the bone 500 tissue, then at least an inner part of the guide 18 has to be sterile. Thus, before screwing the locking screw, the guide 18 has to be inserted into the trocar 12.

Referring now to FIG. 12, schematically illustrating, according to an exemplary embodiment, a lateral see-through view of a bone 500, an intramedullary nail 20 inserted into a medullary canal of the bone 500, a trocar 12 attached to the bone 500, a guide 18 inserted in the trocar 12, and a locking screw 30 screwed into a nail hole 202 of the intramedullary nail 20 through the guide 18. At this stage the locking screw 30 is inserted into the guide 18, and then screwed into the drill made in the bone 500, and further into a nail hole 202 of the intramedullary nail 20 that was previously aligned with the drill in the bone 500, by using the camera.

The present subject matter further provides a method for inserting a locking screw 30 into a nail hole 202 of an intramedullary nail 20 inserted in a medullary canal of a bone 500. It should be noted that the method does not encompass the entire process of intramedullary nailing, but rather relates to the insertion of the locking screw 30 into the nail hole 202 of the intramedullary nail 20 that is inserted into the medullary canal of the bone 500, by using the system 1 of the present subject matter.

According to one embodiment, the method comprises:
providing a bone 500 with a guide wire inserted into a medullary canal of the bone 500, after the medullary canal was expanded;
providing a system 1 for inserting a locking screw 30 into a nail hole 202 of an intramedullary nail 20 inserted into a medullary canal of a bone 500, as described herein;
substantially vertically attaching a trocar 12 to the bone 500 in a location where it is desired to insert a locking screw 30 into a nail hole 202 of an intramedullary nail 20 that is inserted into the intramedullary canal of the bone 500, as illustrated in FIG. 1;
inserting a drill centralizer 14 into the trocar 12, as illustrated in FIG. 2;
puncturing a starter hole in the bone 500 with a centralizer tip 142 of the drill centralizer 14;
removing the drill centralizer 14 from the trocar 12, as illustrated in FIG. 3;
inserting a guide 18 into the trocar 12, as illustrated in FIG. 4;
inserting a drill bit 19 of a drilling device into the guide 18, as illustrated in FIG. 6;
drilling a drill in the bone 500 until the medullary canal of the bone 500;
removing the drill bit 19 from the guide 18;
removing the guide 18 from the trocar 12, as illustrated in FIG. 7;
inserting a sleeve 16 into the trocar 12, as illustrated in FIGS. 8-10;
inserting a camera into the sleeve 16;
acquiring visual light images of the medullary canal through the drill in the bone 500 with the camera;
inserting an intramedullary nail 20 into the medullary canal of the bone 500, through the guide wire that is inserted in the medullary canal, until a nail hole 202 of the intramedullary nail 20 is detected by the camera;
aligning the nail hole 202 of the intramedullary nail 20 with the drill made in the bone 500. This can be achieved, for example, by rotating the intramedullary nail 20 around a longitudinal axis of the intramedullary nail 20;
removing the camera from the sleeve 16;
removing the sleeve 16 from the trocar 12;
inserting a guide 18 into the trocar 12, as illustrated in FIG. 11;
inserting a locking screw 30 into the screw guide 18;
inserting the locking screw into the nail hole 202 of the intramedullary nail 20 that is aligned with the drill in the bone 500, as illustrated in FIG. 12; and
removing the trocar 12, and the screw guide 18 from the bone 500.

According to another embodiment, the nail hole 202 of the intramedullary nail 20 is a distal nail hole 202. Accordingly, the locking screw 30 is firstly inserted into the distal nail hole 202 of the intramedullary nail 20. Then, a locking screw 30 is inserted into a proximal nail hole of the intramedullary nail 20, in order to entirely fix the intramedullary nail 20 in place.

The present subject matter further provide a system 1 for use in inserting a locking screw 30 into a nail hole 202 of an intramedullary nail 20 inserted in a medullary canal of a bone 500, the inserting of the locking screw 30 into the nail hole 202 of the intramedullary nail 20 inserted in the medullary canal of the bone 500, comprising:
providing a bone 500 with a guide wire inserted into a medullary canal of the bone 500, after the medullary canal was expanded;
providing a system 1 for inserting a locking screw 30 into a nail hole 202 of an intramedullary nail 20 inserted into a medullary canal of a bone 500, as described herein;
substantially vertically attaching a trocar 12 to the bone 500 in a location where it is desired to insert a locking screw 30 into a nail hole 202 of an intramedullary nail 20 that is inserted into the intramedullary canal of the bone 500, as illustrated in FIG. 1;
inserting a drill centralizer 14 into the trocar 12, as illustrated in FIG. 2;
puncturing a starter hole in the bone 500 with a centralizer tip 142 of the drill centralizer 14;
removing the drill centralizer 14 from the trocar 12, as illustrated in FIG. 3;
inserting a guide 18 into the trocar 12, as illustrated in FIG. 4;
inserting a drill bit 19 of a drilling device into the guide 18, as illustrated in FIG. 6;
drilling a drill in the bone 500 until the medullary canal of the bone 500;
removing the drill bit 19 from the guide 18;
removing the guide 18 from the trocar 12, as illustrated in FIG. 7;
inserting a sleeve 16 into the trocar 12, as illustrated in FIGS. 8-10;
inserting a camera into the sleeve 16;
acquiring visual light images of the medullary canal through the drill in the bone 500 with the camera;
inserting an intramedullary nail 20 into the medullary canal of the bone 500, through the guide wire that is inserted in the medullary canal, until a nail hole 202 of the intramedullary nail 20 is detected by the camera;
aligning the nail hole 202 of the intramedullary nail 20 with the drill made in the bone 500. This can be achieved, for example, by rotating the intramedullary nail 20 around a longitudinal axis of the intramedullary nail 20;
removing the camera from the sleeve 16;
removing the sleeve 16 from the trocar 12;
inserting a guide 18 into the trocar 12, as illustrated in FIG. 11;
inserting a locking screw 30 into the screw guide 18;
inserting the locking screw into the nail hole 202 of the intramedullary nail 20 that is aligned with the drill in the bone 500, as illustrated in FIG. 12; and
removing the trocar 12, and the screw guide 18 from the bone 500.

According to another embodiment, the nail hole 202 of the intramedullary nail 20 is a distal nail hole 202. Accordingly, the locking screw 30 is firstly inserted into the distal nail hole 202 of the intramedullary nail 20. Then, a locking screw 30 is inserted into a proximal nail hole of the intramedullary nail 20, in order to entirely fix the intramedullary nail 20 in place.

It is appreciated that certain features of the subject matter, which are, for clarity, described in the context of separate embodiments, may also be provided in combination in a single embodiment. Conversely, various features of the subject matter, which are, for brevity, described in the context of a single embodiment, may also be provided separately or in any suitable sub combination.

Although the subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A system for inserting a locking screw into a nail hole of an intramedullary nail inserted in a medullary canal of a bone, the system comprising:
a trocar having a tube like structure that is open at both sides, configured to attach to a bone;
a drill centralizer having an elongated shape and comprising a sharp centralizer tip configured to puncture the bone, wherein the drill centralizer is configured to be inserted into the trocar and puncture the bone and create a starter hole in the bone;
a sleeve having a tube-like structure that is open at both sides, configured to be inserted into the trocar, and guide a camera to a center of a drill drilled in the bone;
a guide having a tube-like structure that is open at both sides, configured to be inserted into the trocar, accommodate a drill bit and guide the drill bit to the starter hole punctured on the surface of the bone, and accommodate the drill bit during the drilling in the bone; and
a drill bit of a drilling device, wherein the drill bit comprising a drill bit tip configured to be in contact with the bone surface at an early stage of drilling, to be accommodated in the start hole punctured in the bone at the beginning of the drilling, wherein the drill bit is configured to drill a hole in the bone when the drill bit tip points toward the bone.

2. The system of claim 1, wherein at least an inner space of the trocar is sterile.

3. The system of claim 1, wherein the drill centralizer is sterile.

4. The system claim 1, wherein at least an inner space of the guide is sterile.

5. The system claim 1, wherein the drill bit is sterile.

6. The system of claim 1, wherein the sleeve is not sterile.

7. The system of claim 1, wherein the sleeve is sterile.

8. The system of claim 1, wherein an inner diameter of the sleeve is fit to a diameter of the camera.

9. The system of claim 1, wherein the sleeve further comprises at least one notch along an inner part of the sleeve, wherein the at last one notch is configured to fit to an at least one protrusion of the camera, and wherein the at least one notch is configured to circularly fix the camera and prevent rotation of the camera around a longitudinal axis of the camera.

10. The system of claim 9, wherein the at least one notch is configured to prevent rotation of the camera around a longitudinal axis of a rod or wire to which the camera is attached.

11. The system of claim 9, wherein the at least one notch is configured to allow sliding of the protrusion of the camera along the notch.

12. The system of claim 9, wherein the at least one notch runs from an external edge of the sleeve, and ends at a distance from an internal edge of the sleeve.

13. The system of claim 12, wherein the distance from an internal edge of the sleeve is configured to prevent contact of the camera with the bone and tissues in the vicinity of the bone.

14. The system of claim 1, wherein the guide is further configured to accommodate a locking screw and guide the locking screw to the drill made in the bone and further into the nail hole that is aligned with the drill made in the bone.

15. A method for inserting a locking screw into a nail hole of an intramedullary nail inserted in a medullary canal of a bone, the method comprising:
providing a bone with a guide wire inserted into a medullary canal of the bone, after the medullary canal was expanded;
providing a system according to claim 1
substantially vertically attaching the trocar to the bone in a location where it is desired to insert a locking screw into a nail hole of an intramedullary nail that is inserted into the intramedullary canal of the bone;
inserting the drill centralizer into the trocar;
puncturing a starter hole in the bone with the centralizer tip of the drill centralizer;
removing the drill centralizer from the trocar;
inserting the guide into the trocar;
inserting the drill bit of the drilling device into the guide;
drilling a drill in the bone until the medullary canal of the bone;
removing the drill bit from the guide;
removing the guide from the trocar;
inserting the sleeve into the trocar;
inserting the camera into the sleeve;
acquiring visual light images of the medullary canal through the drill in the bone with the camera;
inserting the intramedullary nail into the medullary canal of the bone, through the guide wire that is inserted in the medullary canal, until a nail hole of the intramedullary nail is detected by the camera;
aligning the nail hole of the intramedullary nail with the drill made in the bone;
removing the camera from the sleeve;
removing the sleeve from the trocar;
inserting the guide into the trocar;
inserting the locking screw into the screw guide;
inserting the locking screw into the nail hole of the intramedullary nail that is aligned with the drill in the bone; and
removing the trocar, and the screw guide from the bone.

16. The method of claim 15, wherein the aligning the nail hole of the intramedullary nail with the drill made in the bone is achieved by rotating the intramedullary nail around a longitudinal axis of the intramedullary nail.

17. The method of claim 15, wherein the nail hole of the intramedullary nail is a distal nail hole, wherein the locking screw is firstly inserted into the distal nail hole of the intramedullary nail, and then, a locking screw is inserted into a proximal nail hole of the intramedullary nail.

18-20. (canceled)
